(19) United States
(12) Patent Application Publication
(10) Pub. No.: US 2009/0130255 A1
(43) Pub. Date: May 21, 2009

(54) Catalase Gene and Use Thereof

(75) Inventors: Yoshihiro Nakao, Osaka (JP); Yukiko Kadama, Osaka (JP); Tomoko Shimonaga, Osaka (JP); Fumihiko Omura, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, Osaka (JP)

(21) Appl. No.: 11/919,226

(22) PCT Filed: Dec. 22, 2006
(86) PCT No.: PCT/JP2006/326308

§ 371 Date: Oct. 25, 2007

(30) Foreign Application Priority Data
Feb. 28, 2006 (JP) 2006-053898

(51) Int. Cl. C12C 11/00; C12G 1/00; C12P 21/06; C12N 9/02; C12N 1/18; C07H 21/04

(52) U.S. Cl. 426/15; 435/69.1; 435/189; 435/320.1; 435/254.21; 426/16; 536/23.2

(57) ABSTRACT

The present invention relates to a gene encoding a catalase and use thereof, in particular, a brewery yeast having high sulfite-producing capability, alcoholic beverages produced with said yeast, and a method for producing said beverages. More particularly, the present invention relates to a yeast, whose capability of producing sulfite, that contribute to stability of flavor in products, is enhanced by amplifying expression level of CTT1 gene encoding a catalase Ctt1p, especially non-ScCTT1 gene or ScCTT1 gene specific to a lager brewing yeast, and to a method for producing alcoholic beverages with said yeast.

### TECHNICAL FIELD

The present invention relates to a gene encoding catalase and use thereof, in particular, a brewery yeast for producing alcoholic beverages with superior flavor, alcoholic beverages produced with said yeast, and a method for producing said beverages. More particularly, the present invention relates to a yeast, whose capability of producing sulfite that contribute to stability of flavor in products, is enhanced by amplifying expression level of CTT1 gene encoding Ctt1p that is a catalase in a brewery yeast, especially non-ScCTT1 gene or ScCTT1 gene specific to a lager brewing yeast, and to a method for producing alcoholic beverages with said yeast.

### BACKGROUND ART

Sulfite has been known as a compound having high anti-oxidative activity, and thus has been widely used in the fields of food, beverages, pharmaceutical products or the like (for example, Japanese Patent Application Laid-Open Nos. H06-040907 and 2000-093096). In alcoholic beverages, sulfite has been used as an anti-oxidant. For example, because sulfite plays an important role in quality maintenance of wine that needs long-term maturation, addition of up to 350 ppm (parts per million) of residual concentration is permitted by the Ministry of Health, Welfare and Labor in Japan. Further, it is also known that shelf life (quality maintained period) varies depending upon sulfite concentration in a product in beer brewing. Thus, it is quite important to increase the content of this compound from the viewpoint of flavor stability or the like.

The easiest way to increase the sulfite content in a product is addition of sulfite. However, sulfite is treated as a food additive, resulting in some problems such as constraint of product development and negative images of food additives of consumers.

Methods of increasing sulfite content in a fermentation liquor during brewing process include (1) a method based on process control, and (2) a method based on breeding of yeast. In the method based on a process control, since the amount of sulfite produced is in inverse proportion to the amount of initial oxygen supply, supplied amount of oxygen is reduced to increase amount of sulfite produced and to prevent oxidation.

On the other hand, gene manipulation techniques are used in the method based on breeding of yeast. Yeast biosynthesizes sulfur-containing compounds which are required for its biological activity. Sulfite is produced as an intermediate in the biosynthesis of sulfur-containing compounds. Thus, amount of sulfite in products can be increased by utilizing the ability of yeast without adding sulfite from outside.

The MET3 and MET14 are genes encoding reductases participating in steps which are involved in biosynthesis of sulfite from sulfate ion taken from culture medium. Korch et al. attempted to increase a sulfite-producing capability of yeasts by increasing expression level of the two genes, and found that MET14 is more effective (C. Korch et al., Proc. Eur. Brew. Conv. Conger., Lisbon, 201-208, 1991). Also, Hansen et al. attempted to increase production amount of sulfite by disrupting MET10 gene encoding a sulfite ion reductase to prevent reduction of sulfite produced (J. Hansen et al., Nature Biotech., 1587-1591, 1996). On the other hand, however, delay in fermentation or increase in acetaldehyde and 1-propanol, which are undesirable flavor ingredients, are also observed.

Further, Fujimura et al. attempted to increase sulfite content in beer by increasing expression level of a non-ScSSU1 gene unique to a lager brewing yeast among SSU1 genes encoding sulfite ion efflux pump of yeast to promote excretion of sulfite to outside the yeast cells (Fujimura et al., Abstract of 2003 Annual Conference of the Japan Society for Bioscience, Biotechnology and Agrochem., 159, 2003).

### DISCLOSURE OF INVENTION

As mentioned above, the easiest way to increase sulfite content in a product is addition of sulfite. However, it is desirable to minimize use of food additives in view of recent consumers' preference, i.e., avoidance of food additives and preference of natural materials. Thus, it is desirable to achieve sulfite content which is effective level for flavor stability by use of biological activity of yeast itself without adding sulfite from outside. However, the method based on a process control as described above may not be practical since shortage of oxygen may cause decrease in growth rate of yeasts, resulting in delay in fermentation and quality loss.

Further, in breeding of yeast using gene manipulation techniques, there is a report stating that ten times or more sulfite content than parent strain was achieved (J. Hansen et al., Nature Biotech., 1587-1591, 1996). However, there are problems such as delay in fermentation and increase of undesirable flavor ingredients such as acetaldehyde and 1-propanol. Thus, there are problems with the yeast for practical use. Thus, there has been a need for a method for breeding yeast capable of producing sufficient amount of sulfite without impairing the fermentation rates and quality of the products.

To solve the problems described above, the present inventors made extensive studies, and as a result succeeded in identifying and isolating a gene encoding a catalase from a lager brewing yeast. Moreover, a yeast in which the obtained gene was transformed and expressed was produced to confirm increase in production of sulfite, thereby completing the present invention.

Thus, the present invention relates to a catalase gene existing in a lager brewing yeast, to a protein encoded by said gene, to a transformed yeast in which the expression of said gene is controlled, to a method for controlling the amount of sulfite produced in a product by using a yeast in which the expression of said gene is controlled. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO:1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 with one or more amino acids thereof being deleted, substituted, inserted and/or added, and having a catalase activity;

(d) a polynucleotide comprising a polynucleotide encoding a protein having an amino acid sequence having 60% or higher identity with the amino acid sequence of SEQ ID NO:2, and having a catalase activity;

(e) a polynucleotide comprising a polynucleotide which hybridizes to a polynucleotide consisting of a nucleotide sequence complementary to the nucleotide sequence of SEQ ID NO:1 under stringent conditions, and which encodes a protein having a catalase activity; and

(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having a catalase activity.

(2) The polynucleotide of (1) above selected from the group consisting of:

(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has a catalase activity;

(h) a polynucleotide encoding a protein having 90% or higher identity with the amino acid sequence of SEQ ID NO: 2, and having a catalase activity; and

(i) a polynucleotide which hybridizes to SEQ ID NO: 1 or which hybridizes to a nucleotide sequence complementary to the nucleotide sequence of SEQ ID NO: 1 under high stringent conditions, and which encodes a protein having a catalase activity.

(3) The polynucleotide of (1) above comprising a polynucleotide consisting of SEQ ID NO: 1.

(4) The polynucleotide of (1) above comprising a polynucleotide encoding a protein consisting of SEQ ID NO: 2.

(5) The polynucleotide of any one of (1) to (4) above, wherein the polynucleotide is DNA.

(6) A protein encoded by the polynucleotide of any one of (1) to (5) above.

(7) A vector comprising the polynucleotide of any one of (1) to (5) above.

(7a) The vector of (7) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(8) A vector comprising the polynucleotide selected from the group consisting of:

(j) a polynucleotide encoding a protein consisting of the amino acid sequence of SEQ ID NO: 4, or encoding an amino acid sequence of SEQ ID NO: 4 wherein 1 to 10 amino acids thereof is deleted, substituted, inserted, and/or added, and wherein said protein has a catalase activity;

(k) a polynucleotide encoding a protein having 90% or higher identity with the amino acid sequence of SEQ ID NO: 4, and having a catalase activity; and

(l) a polynucleotide which hybridizes to SEQ ID NO: 3 or which hybridizes to a nucleotide sequence complementary to the nucleotide sequence of SEQ ID NO: 3 under high stringent conditions, and which encodes a protein having a catalase activity.

(9) A yeast, wherein the vector of (7) or (8) above is introduced.

(10) The yeast of (9) above, wherein a sulfite-producing capability is enhanced.

(11) The yeast of (10) above, wherein a sulfite-producing capability is enhanced by increasing an expression level of the protein of (6) above.

(12) A method for producing an alcoholic beverage comprising culturing the yeast of any one of (9) to (11) above.

(13) The method for producing an alcoholic beverage of (12) above, wherein the brewed alcoholic beverage is a malt beverage.

(14) The method for producing an alcoholic beverage of (12) above, wherein the brewed alcoholic beverage is wine.

(15) An alcoholic beverage produced by the method of any one of (12) to (14) above.

(16) A method for assessing a test yeast for its sulfite-producing capability, comprising using a primer or a probe designed based on a nucleotide sequence of a gene having the nucleotide sequence of SEQ ID NO: 1 or SEQ ID NO: 3, and encoding a protein having a catalase activity.

(16a) A method for selecting a yeast having an enhanced sulfite-producing capability by using the method described in (16) above.

(16b) A method for producing an alcoholic beverage (for example, beer) by using the yeast selected with the method in (16a) above.

(17) A method for assessing a test yeast for its sulfite-producing capability, comprising: culturing a test yeast; and measuring an expression level of a gene having the nucleotide sequence of SEQ ID NO: 1 or SEQ ID NO: 3, and encoding a protein having a catalase activity.

(17a) A method for selecting a yeast having a high sulfite-producing capability, which comprises assessing a test yeast by the method described in (17) above and selecting a yeast having a high expression level of a gene encoding a protein having a catalase activity.

(17b) A method for producing an alcoholic beverage (for example, beer) by using the yeast selected with the method in (17a) above.

(18) A method for selecting a yeast, comprising: culturing test yeasts; quantifying the protein according to (6) or measuring an expression level of a gene having the nucleotide sequence of SEQ ID NO: 1 or SEQ ID NO: 3, and encoding a protein having a catalase activity; and selecting a test yeast having said protein amount or said gene expression level according to a target sulfite-producing capability.

(19) The method for selecting a yeast according to (18) above, comprising: culturing a reference yeast and test yeasts; measuring an expression level of a gene having the nucleotide sequence of SEQ ID NO: 1 or SEQ ID NO: 3, and encoding a protein having a catalase activity in each yeast; and selecting a test yeast having the gene expressed higher than that in the reference yeast.

(20) The method for selecting a yeast according to (18) above, comprising: culturing a reference yeast and test yeasts; quantifying the protein according to (6) above in each yeast; and selecting a test yeast having said protein in a larger amount than that in the reference yeast.

(21) A method for producing an alcoholic beverage comprising: conducting fermentation for producing an alcoholic beverage using the yeast according to any one of (9) to (11) above or a yeast selected by the method according to any one of (18) to (20) above; and adjusting sulfite concentration.

According to the method for producing alcoholic beverages of the present invention, the content of sulfite which has an anti-oxidative activity in products can be increased so that alcoholic beverages which have superior stability of flavor and longer shelf life can be produced.

### BRIEF DESCRIPTION OF DRAWINGS

FIG. 1 shows the cell growth with time upon beer fermentation test. The horizontal axis represents fermentation time while the vertical axis represents optical density at 660 nm (OD660).

FIG. 2 shows the extract consumption with time upon beer fermentation test. The horizontal axis represents fermentation time while the vertical axis represents apparent extract concentration (w/w %).

FIG. 3 shows the expression behavior of non-ScCTT1 gene in yeasts upon beer fermentation test. The horizontal axis represents fermentation time while the vertical axis represents the brightness of detected signal.

FIG. 4 shows the cell growth with time upon beer fermentation test using the non-ScCTT1-highly expressed strain. The horizontal axis represents fermentation time while the vertical axis represents optical density at 660 nm (OD660).

FIG. 5 shows the extract consumption with time upon beer fermentation test using the non-ScCTT1-highly expressed strain. The horizontal axis represents fermentation time while the vertical axis represents apparent extract concentration (w/w %).

FIG. 6 shows sulfite concentration in the fermentation broth (at the completion of fermentation) during beer fermentation test using the non-ScCTT1-highly expressed strain.

FIG. 7 shows the cell growth with time upon beer fermentation test. The horizontal axis represents fermentation time while the vertical axis represents optical density at 660 nm (OD660).

FIG. 8 shows the extract consumption with time upon beer fermentation test. The horizontal axis represents fermentation time while the vertical axis represents apparent extract concentration (w/w %).

FIG. 9 shows the expression behavior of ScCTT1 gene in yeasts upon beer fermentation test. The horizontal axis represents fermentation time while the vertical axis represents the brightness of detected signal.

FIG. 10 shows the cell growth with time upon beer fermentation test using the ScCTT1-highly expressed strain. The horizontal axis represents fermentation time while the vertical axis represents optical density at 660 nm (OD660).

FIG. 11 shows the extract consumption with time upon beer fermentation test using the ScCTT1-highly expressed strain. The horizontal axis represents fermentation time while the vertical axis represents apparent extract concentration (w/w %).

FIG. 12 shows sulfite concentration in the fermentation broth (at the completion of fermentation) during beer fermentation test using the ScCTT1-highly expressed strain.

### BEST MODES FOR CARRYING OUT THE INVENTION

The present inventors isolated and identified non-ScCTT1 gene encoding a protein having a catalase activity unique to lager brewing yeast based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2. Furthermore, the present inventors isolated and identified ScCTT1 gene encoding a protein having a catalase activity unique to lager brewing yeast. The nucleotide sequence of the gene is represented by SEQ ID NO: 3. Further, an amino acid sequence of a protein encoded by the gene is represented by SEQ ID NO: 4.

### 1. Polynucleotide of the Invention

First of all, the present invention provides (a) a polynucleotide comprising a polynucleotide of the nucleotide sequence of SEQ ID NO:1 or SEQ ID NO: 3; and (b) a polynucleotide comprising a polynucleotide encoding a protein of the amino acid sequence of SEQ ID NO:2 or SEQ ID NO: 4. The polynucleotide can be DNA or RNA.

The target polynucleotide of the present invention is not limited to the polynucleotide encoding a protein having a catalase activity derived from lager brewing yeast and may include other polynucleotides encoding proteins having equivalent functions to said protein. Proteins with equivalent functions include, for example, (c) a protein of an amino acid sequence of SEQ ID NO: 2 or SEQ ID NO: 4 with one or more amino acids thereof being deleted, substituted, inserted and/or added and having a catalase activity.

Such proteins include a protein consisting of an amino acid sequence of SEQ ID NO: 2 or SEQ ID NO: 4 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having a catalase activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO: 2 or SEQ ID NO: 4, and having a catalase activity. In general, the percentage identity is preferably higher.

The catalase activity can be assessed, for example by, a method of Osorio et al., Archives of Microbiology, 181(3), 231-236 (2004).

Furthermore, the present invention also contemplates (e) a polynucleotide comprising a polynucleotide which hybridizes to a polynucleotide consisting of a nucleotide sequence complementary to the nucleotide sequence of SEQ ID NO: 1 or SEQ ID NO: 3 under stringent conditions and which encodes a protein having a catalase activity; and (f) a polynucleotide comprising a polynucleotide which hybridizes to a polynucleotide complementary to a nucleotide sequence of encoding a protein of SEQ ID NO: 2 or SEQ ID NO: 4 under stringent conditions, and which encodes a protein having a catalase activity.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or SEQ ID NO: 3 or polynucleotide encoding the amino acid sequence of SEQ ID NO: 2 or SEQ ID NO: 4 as a probe. The hybridization method may be a method described, for example, in Molecular Cloning 3rd Ed., Current Protocols in Molecular Biology, John Wiley & Sons 1987-1997.

The term “stringent conditions” as used herein may be any of low stringency conditions, moderate stringency conditions or high stringency conditions. “Low stringency conditions” are, for example, 5×SSC, 5× Denhardt's solution, 0.5% SDS, 50% formamide at 32° C. “Moderate stringency conditions” are, for example, 5×SSC, 5× Denhardt's solution, 0.5% SDS, 50% formamide at 42° C. “High stringency conditions” are, for example, 5×SSC, 5× Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2 or SEQ ID NO: 4 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (l) above. A preferred protein of the present invention comprises an amino acid sequence of SEQ ID NO:2 or SEQ ID NO: 4 with one or several amino acids thereof being deleted, substituted, inserted and/or added, and has a catalase activity.

Such protein includes those having an amino acid sequence of SEQ ID NO: 2 or SEQ ID NO: 4 with amino acid residues thereof of the number mentioned above being deleted, substituted, inserted and/or added and having a catalase activity. In addition, such protein includes those having homology as described above with the amino acid sequence of SEQ ID NO: 2 or SEQ ID NO: 4 and having a catalase activity.

Such proteins may be obtained by employing site-directed mutation described, for example, in Molecular Cloning 3rd Ed., Current Protocols in Molecular Biology, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides (DNA) described in (a) to (l) above. Generally, the vector of the present invention comprises an expression cassette including as components (x) a promoter that can transcribe in a yeast cell; (y) a polynucleotide (DNA) described in any of (a) to (l) above that is linked to the promoter in sense or antisense direction; and (z) a signal that functions in the yeast with respect to transcription termination and polyadenylation of RNA molecule.

A vector introduced in the yeast may be any of a multicopy type (YEp type), a single copy type (YCp type), or a chromosome integration type (YIp type). For example, YEp24 (J. R. Broach et al., Experimental Manipulation of Gene Expression, Academic Press, New York, 83, 1983) is known as a YEp type vector, YCp50 (M. D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the brewery yeast and they have no influence on the concentration of constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake. Specifically, yeasts such as genus Saccharomyces may be used. According to the present invention, a lager brewing yeast, for example, Saccharomyces pastorianus W34/70, Saccharomyces carlsbergensis NCYC453 or NCYC456, or Saccharomyces cerevisiae NBRC1951, NBRC1952, NBRC1953 or NBRC1954 may be used. In addition, wine yeasts such as wine yeasts #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929(1978)), a lithium acetate method (J. Bacteriology, 153: 163 (1983)), and methods described in Proc. Natl. Acad. Sci. USA, 75: 1929 (1978), Methods in Yeast Genetics, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York, 117(1979)), etc.) such that OD600 nm will be 1 to 6. This culture yeast is collected by centrifugation, washed and pre-treated with alkali ion metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in Molecular Cloning 3rd Ed., and Methods in Yeast Genetics, A Laboratory Manual (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to increase content of sulfite of desired alcoholic beverages with superior stability of flavor. In addition, yeasts to be selected by the yeast assessment method of the present invention described below can also be used. The target alcoholic beverages include, for example, but not limited to beer, sparkling liquor (happoushu) such as a beer-taste beverage, wine, sake and the like.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages with increased content of sulfite. Thus, according to the present invention, alcoholic beverages with superior stability of flavor can be produced using the existing facility without increasing the cost.

### 5. Yeast Assessment Method of the Invention

The present invention relates to a method for assessing a test yeast for its capability of producing sulfite by using a primer or a probe designed based on a nucleotide sequence of a gene having the nucleotide sequence of SEQ ID NO:1 or SEQ ID NO: 3, and encoding a protein having a catalase activity. General techniques for such assessment method are known and are described in, for example, WO01/040514, Japanese Laid-Open Patent Application No. 8-205900 or the like. This assessment method is described in below.

First, genome of a test yeast is prepared. For this preparation, any known method such as Hereford method or potassium acetate method may be used (e.g., Methods in Yeast Genetics, Cold Spring Harbor Laboratory Press, 130 (1990)). Using a primer or a probe designed based on a nucleotide sequence (preferably, ORF sequence) of the gene encoding a protein having a catalase activity, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the capability of producing sulfite of the yeast as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the gene having the nucleotide sequence of SEQ ID NO: 1 or SEQ ID NO: 3 and encoding a protein having a catalase activity to assess the test yeast for its capability of producing sulfite. In measuring an expression level of the gene encoding a protein having a catalase activity, the test yeast is cultured, and then mRNA or a protein resulting from the gene encoding a protein having a catalase activity, is quantified. The quantification of mRNA or protein may be carried out by employing a known technique. For example, mRNA may be quantified, by Northern hybridization or quantitative RT-PCR, while protein may be quantified, for example, by Western blotting (Current Protocols in Molecular Biology, John Wiley & Sons 1994-2003). Further, expression level of the above-identified gene of the test yeast may be projected by measuring sulfite concentration of fermentation broth obtained after fermentation of the test yeast.

Furthermore, test yeasts are cultured and expression levels of the gene having the nucleotide sequence of SEQ ID NO: 1 or SEQ ID NO: 3, and encoding a protein having a catalase activity are measured to select a test yeast with the gene expression level according to the target catalase activity, thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the gene having the nucleotide sequence of SEQ ID NO: 1 or SEQ ID NO: 3 and encoding a protein having a catalase activity, is measured in each yeast. By selecting a test yeast with the gene expressed higher than that in the reference yeast, a yeast suitable for brewing alcoholic beverages can be selected.

Alternatively, test yeasts are cultured and a yeast with a higher catalase activity is selected, thereby selecting a yeast suitable for brewing desired alcoholic beverages.

In these cases, the test yeasts or the reference yeast may be, for example, a yeast introduced with the vector of the invention, an artificially mutated yeast or a naturally mutated yeast. The catalase activity can be assessed, for example by, a method of Osorio et al., Archives of Microbiology, 181(3), 231-236 (2004). The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g., Yasuji Oshima Ed., Biochemistry Experiments vol. 39, Yeast Molecular Genetic Experiments, pp. 67-75, JSSP).

In addition, examples of yeasts used as the reference yeast or the test yeasts include any yeasts that can be used for brewing, for example, brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954 may be used. Further, whisky yeasts such as Saccharomyces cerevisiae NCYC90; wine yeasts such as wine yeasts #1, 3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Gene Encoding Catalase of Lager Brewing Yeast (non-ScCTT1)

A gene encoding a catalase (non-ScCTT1 gene; SEQ ID NO: 1) specific to a lager brewing yeast was found, as a result of a search utilizing the comparison database described in Japanese Patent Application Laid-Open No. 2004-283169. Based on the acquired nucleotide sequence information, primers non-ScCTT1_for (SEQ ID NO: 5) and non-ScCTT1_rv (SEQ ID NO: 6) were designed to amplify the full-length genes, respectively. PCR was carried out-using chromosomal DNA of a genome sequencing strain, Saccharomyces pastorianus Weihenstephan 34/70 strain (also sometimes referred to as “W34/70 strain”), as a template to obtain DNA fragments including the full-length gene of non-ScCTT1.

The thus-obtained non-ScCTT1 gene fragment was inserted into pCR2.1-TOPO vector (manufactured by Invitrogen Corporation) by TA cloning. The nucleotide sequences of non-ScCTT1 gene were analyzed according to Sanger's method (F. Sanger, Science, 214: 1215, 1981) to confirm the nucleotide sequence.

### Example 2

### Analysis of Expression of non-ScCTT1 Gene during Beer Fermentation

A beer fermentation test was conducted using a lager brewing yeast, Saccharomyces pastorianus 34/70 strain and then mRNA extracted from yeast cells during fermentation was analyzed by a yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

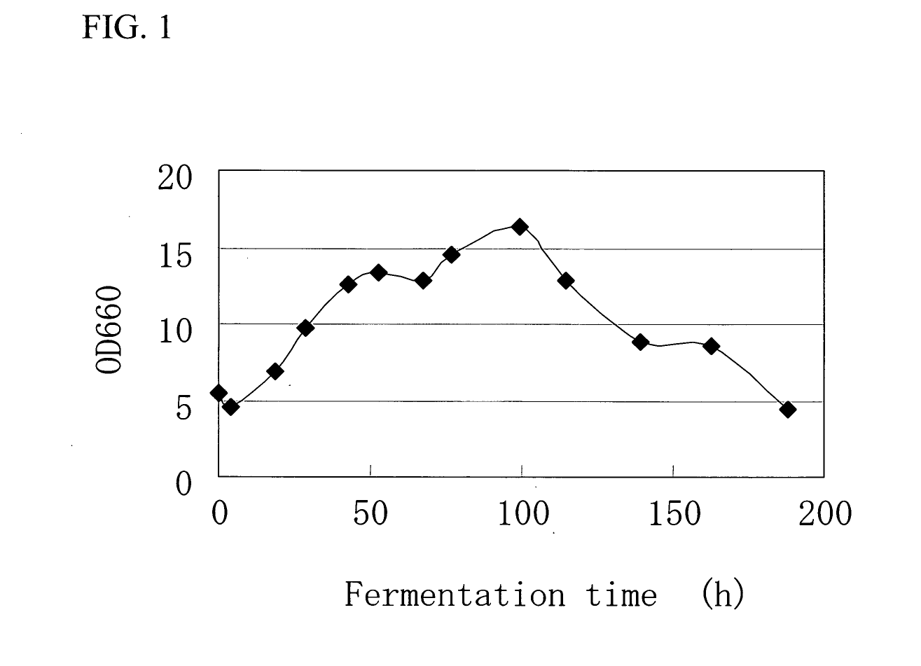

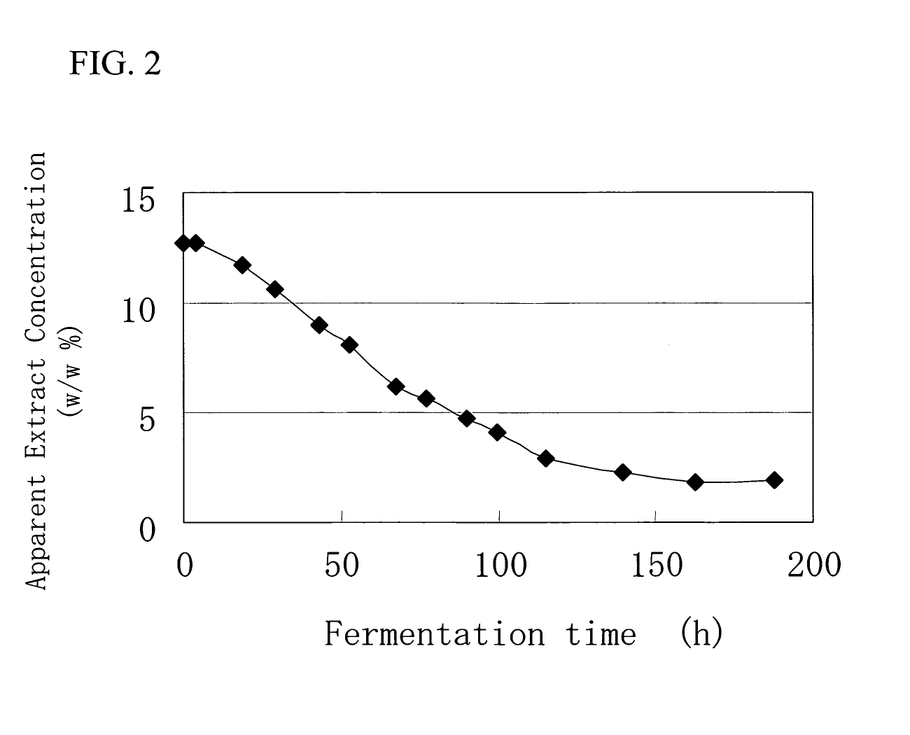

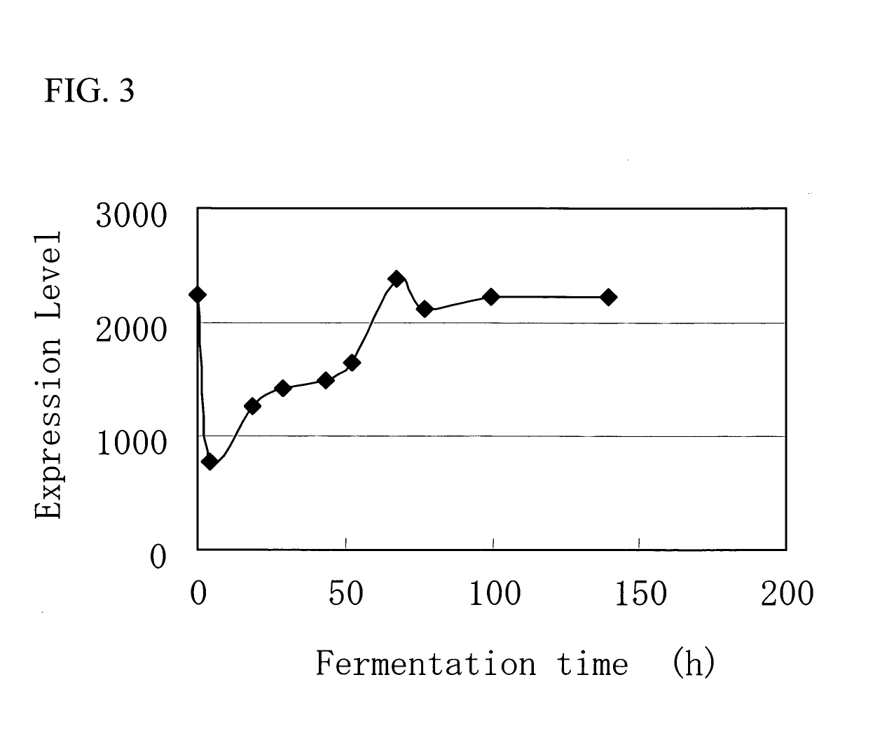

Sampling of fermentation liquid was performed with time, and variation with time of yeast growth amount (FIG. 1) and apparent extract concentration (FIG. 2) was observed. Simultaneously, yeast cells were sampled to prepare mRNA, and the prepared mRNA was labeled with biotin and was hybridized to a beer yeast DNA microarray. The signal was detected using GCOS; GeneChip Operating Software 1.0 (manufactured by Affymetrix Co.). Expression pattern of non-ScCTT1 gene is shown in FIG. 3. As a result, it was confirmed that non-ScCTT1 gene was expressed in the general beer fermentation.

### Example 3

### Preparation of non-ScCTT1 Gene-Highly Expressed Strain

The non-ScCTT1/pCR2.1-TOPO described in Example 1 was digested with restriction enzymes SacI and NotI to prepare a DNA fragment including non-ScCTT1 gene. This fragment was linked to pUP3GLP2 treated with restriction enzymes SacI and NotI, thereby constructing a non-ScCTT1 high expression vector, pUP-nonScCTT1. The yeast expression vector, pUP3GLP2, is a YIp type (chromosome integration type) yeast expression vector having orotidine-5-phosphoric acid decarboxylase gene URA3 at the homologous recombinant site. The introduced gene was highly expressed by the promoter and terminator of glycerylaldehyde-3-phosphoric acid dehydrogenase gene, TDH3. Drug-resistant gene YAP1 as a selective marker for yeast was introduced under the control of the promoter and terminator of galactokinase GAL1, whereby the expression is induced in a culture media comprising galactose. Ampicillin-resistant gene Amp^{r} as a selective marker for E. coli was also included.

Using the high expression vector prepared by the above method, Saccharomyces pastorianus Weihenstephan 164 strain was transformed by the method described in Japanese Patent Application Laid-Open No. 07-303475. A cerulenin-resistant strain was selected in a YPGal plate medium (1% yeast extract, 2% polypeptone, 2% galactose, 2% agar) containing 1.0 mg/L of cerulenin.

### Example 4

### Analysis of Amount of Sulfite Produced during Beer Fermentation

The parent strain and non-ScCTT1-highly expressed strain obtained in Example 3 were used to carry out fermentation test under the following conditions.

| | |
|---|---|
| Wort extract concentration | 12.78% |
| Wort content | 2 L |
| Wort dissolved oxygen concentration | approximately 8 ppm |
| Fermentation temperature | 15° C., constant |
| Yeast pitching rate | 10.5 g wet yeast cells/2 L Wort |

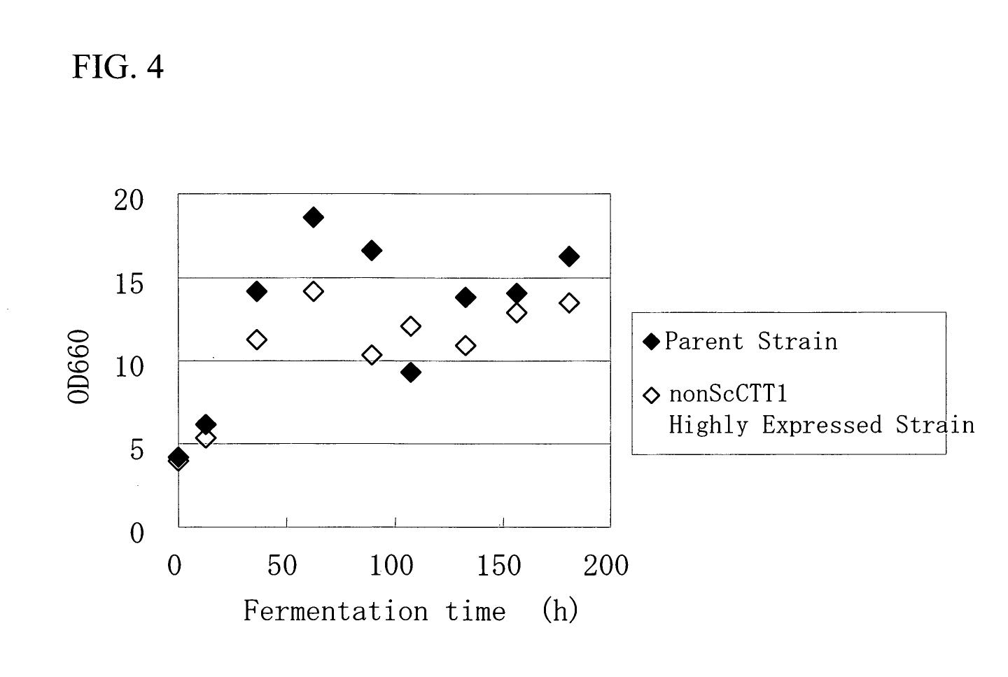

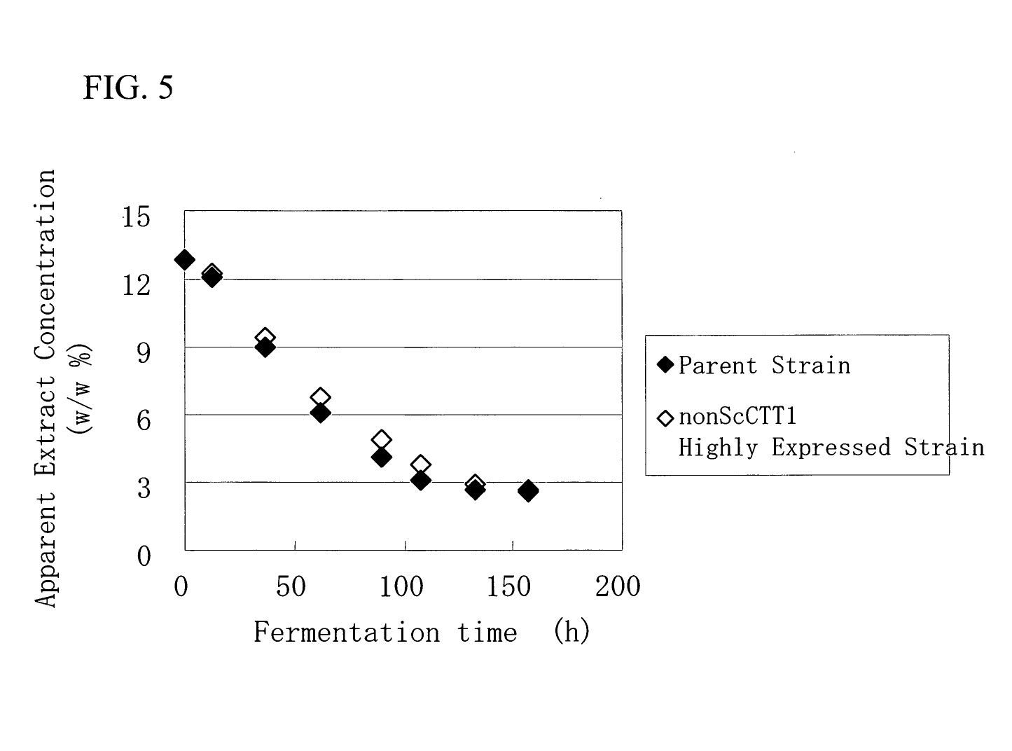

The fermentation broth was sampled with time to observe the cell growth (OD660) (FIG. 4) and extract consumption with time (FIG. 5). Quantification of sulfite concentration at completion of fermentation was carried out by collecting sulfite into hydrogen peroxide aqueous solution by distillation under acidic condition, and titration with alkali (Revised BCOJ Beer Analysis Method by the Brewing Society of Japan).

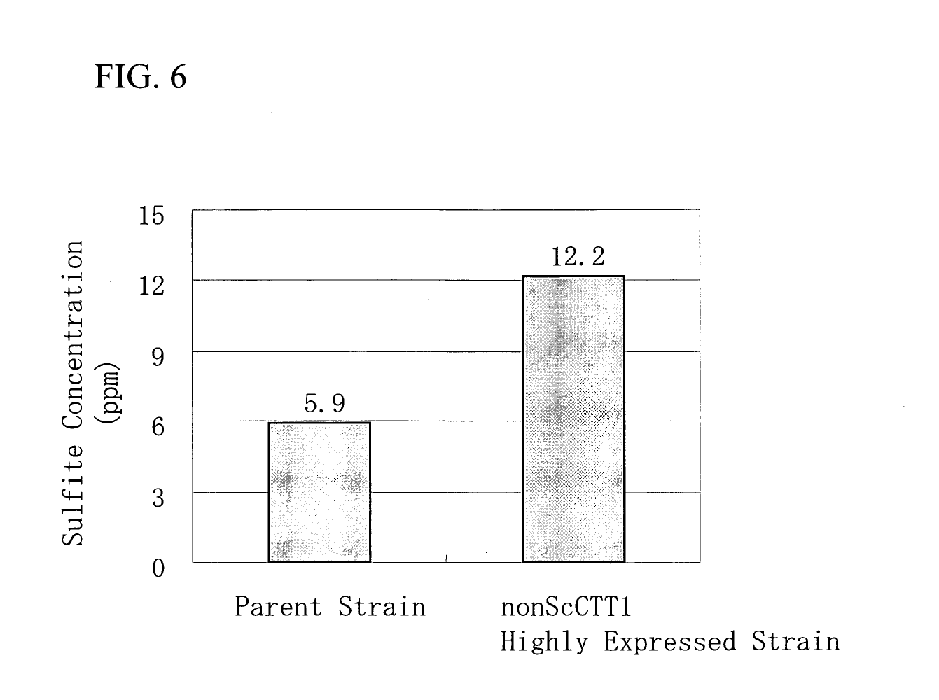

As shown in FIG. 6, the non-ScCTT1-highly expressed strain produced sulfite approximately 2.1 times greater than the parent strain. In addition, significant differences were not observed between the parent strain and the highly expressed strain in cell growth and extract consumption in this testing.

### Example 5

### Cloning of Gene Encoding Catalase (ScCTT1)

A gene encoding a catalase (ScCTT1 gene; SEQ ID NO: 3) specific to a lager brewing yeast was found, as a result of a search utilizing the comparison database described in Japanese Patent Application Laid-Open No. 2004-283169. Based on the acquired nucleotide sequence information, primers ScCTT1_for (SEQ ID NO: 7) and ScCTT1_rv (SEQ ID NO: 8) were designed to amplify the full-length genes, respectively. PCR was carried out using chromosomal DNA of a genome sequencing strain, Saccharomyces pastorianus Weihenstephan 34/70 strain, as a template to obtain DNA fragments including the full-length gene of ScCTT1.

The thus-obtained ScCTT1 gene fragment was inserted into pCR2.1-TOPO vector (manufactured by Invitrogen Corporation) by TA cloning. The nucleotide sequences of ScCTT1 gene were analyzed according to Sanger's method (F. Sanger, Science, 214: 1215, 1981) to confirm the nucleotide sequence.

### Example 6

### Analysis of Expression of ScCTT1 Gene during Beer Fermentation

A beer fermentation test was conducted using a lager brewing yeast, Saccharomyces pastorianus 34/70 strain and then mRNA extracted from yeast cells during fermentation was analyzed by a yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

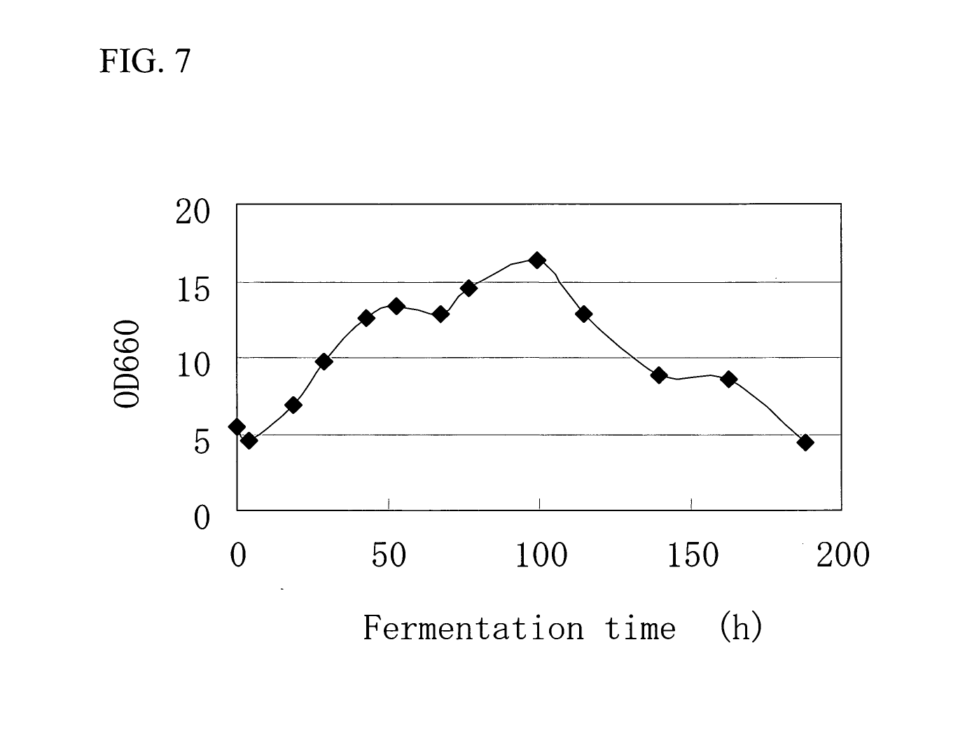

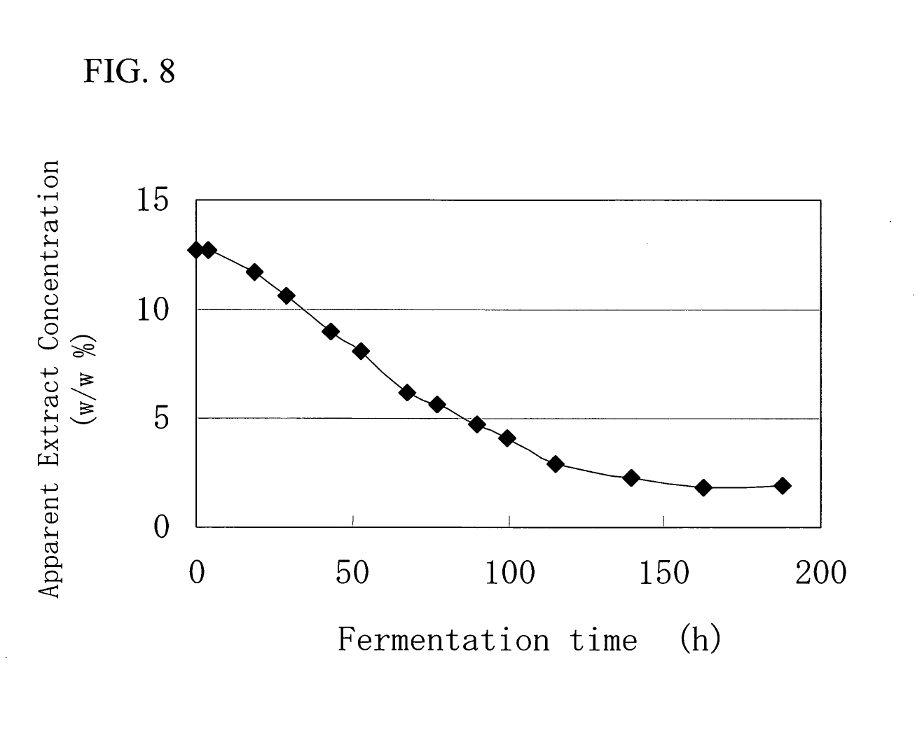

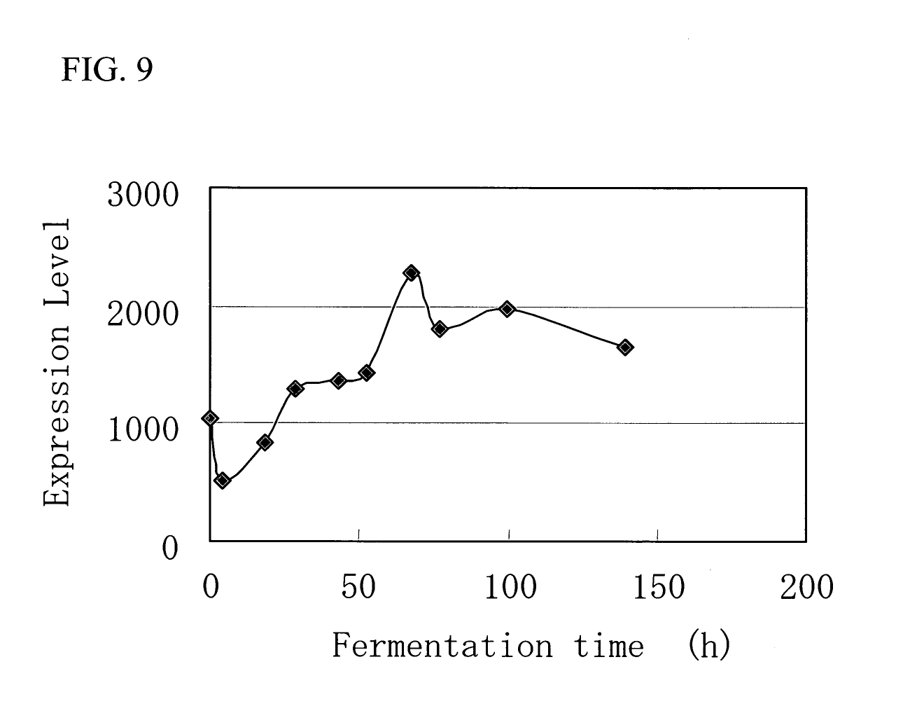

Sampling of fermentation liquid was performed with time, and variation with time of yeast growth amount (FIG. 7) and apparent extract concentration (FIG. 8) was observed. Simultaneously, yeast cells were sampled to prepare mRNA, and the prepared mRNA was labeled with biotin and was hybridized to a beer yeast DNA microarray. The signal was detected using GCOS; GeneChip Operating Software 1.0 (manufactured by Affymetrix Co.). Expression pattern of ScCTT1 gene is shown in FIG. 9. As a result, it was confirmed that ScCTT1 gene was expressed in the general beer fermentation.

### Example 7

### Preparation of ScCTT1-Highly Expressed Strain

The ScCTT1/pCR2.1-TOPO described in Example 5 was digested with restriction enzymes SacI and NotI to prepare a DNA fragment including ScCTT1 gene. This fragment was linked to pUP3GLP2 treated with restriction enzymes SacI and NotI, thereby constructing a ScCTT1 high expression vector, pUP-ScCTT1.

Using the high expression vector prepared by the above method, Saccharomyces pasteurianus Weihenstephaner 164 strain was transformed by the method described in Japanese Patent Application Laid-open No. H7-303475. A cerulenin-resistant strain was selected in a YPGal plate medium (1% yeast extract, 2% polypeptone, 2% galactose, 2% agar) containing 1.0 mg/L of cerulenin.

### Example 8

### Analysis of Amount of Sulfite Produced during Beer Fermentation

The parent strain and ScCTT1-highly expressed strain obtained in Example 7, are used to carry out fermentation test under the following conditions.

| | |
|---|---|
| Wort extract concentration | 12.78% |
| Wort content | 2 L |
| Wort dissolved oxygen concentration | approximately 8 ppm |
| Fermentation temperature | 15° C., constant |
| Yeast pitching rate | 10.5 g wet yeast cells/2 L Wort |

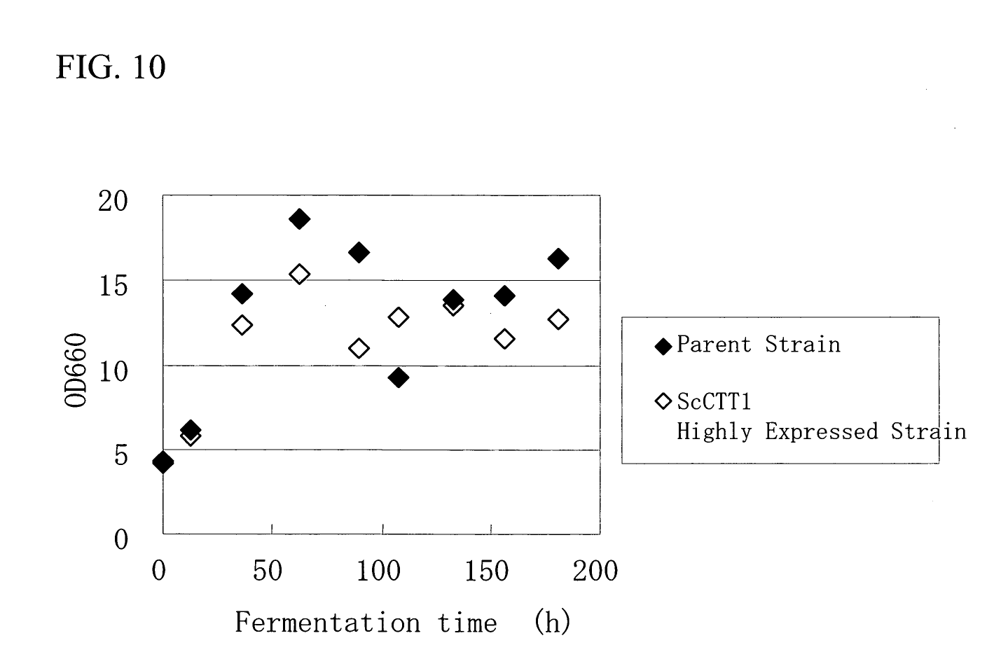

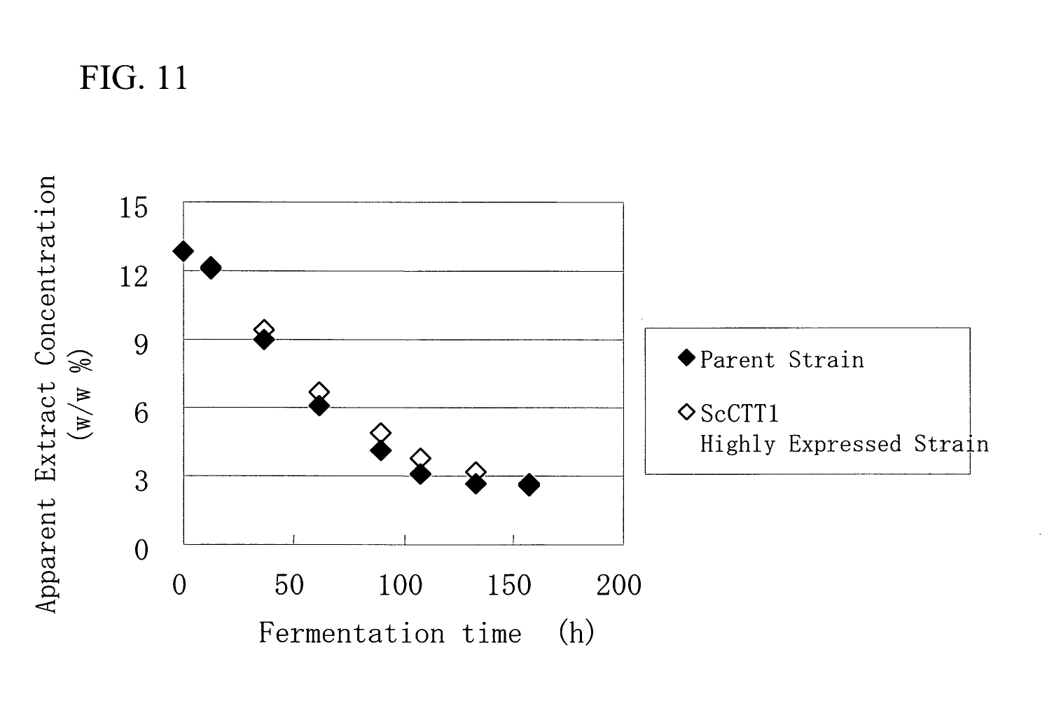

The fermentation broth was sampled with time to observe the cell growth (OD660) (FIG. 10) and extract consumption with time (FIG. 11). Quantification of sulfite concentration at completion of fermentation was carried out by collecting sulfite into hydrogen peroxide aqueous solution by distillation under acidic condition, and titration with alkali (Revised BCOJ Beer Analysis Method by the Brewing Society of Japan).

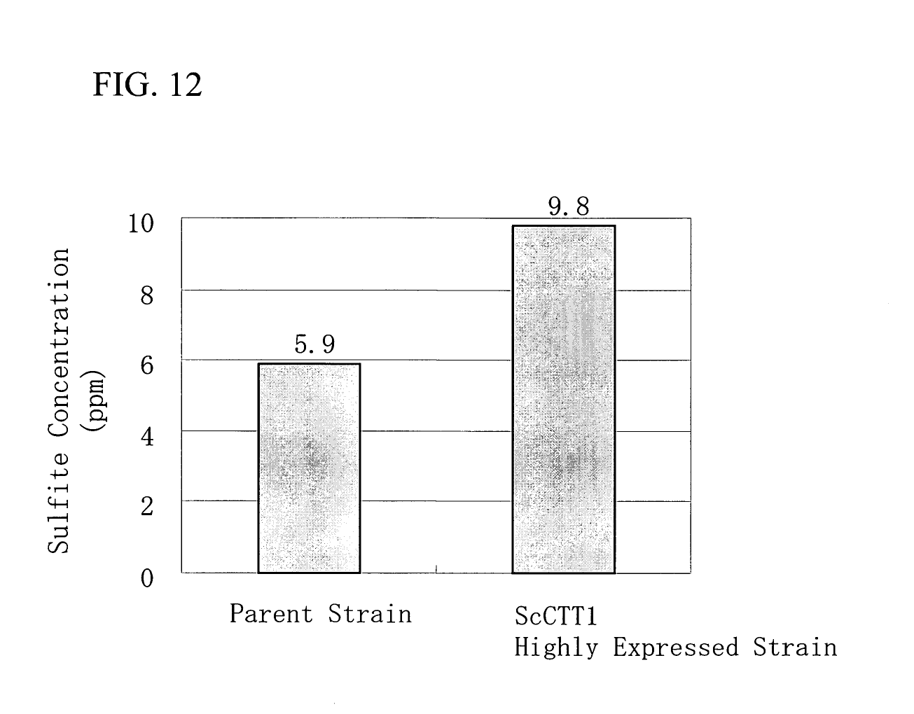

As shown in FIG. 12, the ScCTT1-highly expressed strain produced sulfite approximately 1.7 times greater than the parent strain. In addition, significant differences were not observed between the parent strain and the highly expressed strain in cell growth and extract consumption in this testing.

### INDUSTRIAL APPLICABILITY

According to the method for producing alcoholic beverages of the present invention, because of increase in amount of sulfite which has an anti-oxidative effect in products, alcoholic beverages with superior flavor stability and longer shelf life can be produced.

1. A polynucleotide selected from the group consisting of:
(a) a polynucleotide comprising a polynucleotide consisting of the nucleotide sequence of SEQ ID NO: 1;
(b) a polynucleotide comprising a polynucleotide encoding a protein consisting of the amino acid sequence of SEQ ID NO: 2;
(c) a polynucleotide comprising a polynucleotide encoding a protein consisting of the amino acid sequence of SEQ ID NO: 2 with one or more amino acids thereof being deleted, substituted, inserted and/or added, and having an catalase activity;
(d) a polynucleotide comprising a polynucleotide encoding a protein having an amino acid sequence having 60% or higher identity with the amino acid sequence of SEQ ID NO: 2, and having a catalase activity;
(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a catalase activity; and
(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO: 2 under stringent conditions, and which encodes a protein having a catalase activity.

2. The polynucleotide of claim 1 selected from the group consisting of:
(a) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has a catalase activity;
(b) a polynucleotide encoding a protein having 90% or higher identity with the amino acid sequence of SEQ ID NO: 2, and having a catalase activity; and
(c) a polynucleotide which hybridizes to SEQ ID NO: 1 or which hybridizes to a nucleotide sequence complementary to the nucleotide sequence of SEQ ID NO: 1 under high stringent conditions, and which encodes a protein having a catalase activity.

3. The polynucleotide of claim 1 comprising a polynucleotide consisting of SEQ ID NO: 1.

4. The polynucleotide of claim 1 comprising a polynucleotide encoding a protein consisting of SEQ ID NO: 2.

5. The polynucleotide of claim 1, wherein the polynucleotide is DNA.

6. A protein encoded by the polynucleotide of claim 1.

7. A vector comprising the polynucleotide of claim 1.

8. A vector comprising the polynucleotide selected from the group consisting of:
(a) a polynucleotide encoding a protein consisting of the amino acid sequence of SEQ ID NO: 4, or encoding an amino acid sequence of SEQ ID NO: 4 wherein 1 to 10 amino acids thereof is deleted, substituted, inserted, and/or added, and wherein said protein has a catalase activity;
(b) a polynucleotide encoding a protein having 90% or higher identity with the amino acid sequence of SEQ ID NO: 4, and having a catalase activity; and
(c) a polynucleotide which hybridizes to SEQ ID NO: 3 or which hybridizes to a nucleotide sequence complementary to the nucleotide sequence of SEQ ID NO: 3 under high stringent conditions, and which encodes a protein having a catalase activity.

9. A yeast, wherein the vector of claim 7 is introduced.

10. The yeast of claim 9, wherein a sulfite-producing capability is enhanced.

11. A yeast, wherein a sulfite-producing capability is enhanced by increasing an expression level of the protein of claim 6.

12. A method for producing an alcoholic beverage comprising culturing the yeast of claim 9.

13. The method for producing an alcoholic beverage of claim 12, wherein the brewed alcoholic beverage is a malt beverage.

14. The method for producing an alcoholic beverage of claim 12, wherein the brewed alcoholic beverage is wine.

15. An alcoholic beverage produced by the method of claim 12.

16. A method for assessing a test yeast for its sulfite-producing capability, comprising using a primer or a probe designed based on a nucleotide sequence of a gene having the nucleotide sequence of SEQ ID NO: 1 or SEQ ID NO: 3, and encoding a protein having a catalase activity.

17. A method for assessing a test yeast for its sulfite-producing capability, comprising: culturing a test yeast; and measuring an expression level of a gene having the nucleotide sequence of SEQ ID NO: 1 or SEQ ID NO: 3, and encoding a protein having a catalase activity.

18. A method for selecting a yeast, comprising: culturing test yeasts; quantifying the protein according to claim 6 or measuring an expression level of a gene having the nucleotide sequence of SEQ ID NO: 1 or SEQ ID NO: 3, and encoding a protein having a catalase activity; and selecting a test yeast having said protein amount or said gene expression level according to a target sulfite-producing capability.

19. The method for selecting a yeast according to claim 18, comprising: culturing a reference yeast and test yeasts; measuring an expression level of a gene having the nucleotide sequence of SEQ ID NO: 1 or SEQ ID NO: 3, and encoding a protein having a catalase activity in each yeast; and selecting a test yeast having the gene expressed higher than that in the reference yeast.

20. The method for selecting a yeast according to claim 18, comprising: culturing a reference yeast and test yeasts; quantifying the protein encoded by the polynucleotide in each yeast; and selecting a test yeast having said protein in a larger amount than that in the reference yeast.

21. A method for producing an alcoholic beverage comprising:
(a) conducting fermentation for producing an alcoholic beverage using the yeast according to claim 9; or
(b) a yeast selected by the method comprising: culturing test yeasts; quantifying the protein encoded by the polynucleotide or measuring an expression level of a gene having the nucleotide sequence of SEQ ID NO: 1 or SEQ ID NO: 3, and encoding a protein having a catalase activity; and selecting a test yeast having said protein amount or said gene expression level according to a target sulfite-producing capability; and
(c) adjusting sulfite concentration.


SEQUENCE LISTING (8 sequences)

SEQ ID NO: 1 - DNA, 1683 bp, Saccharomyces sp.
atgtttggtaaaaaggaagaaaagcctgagaaagtttacactttgcaaaatgggttcccatactctcatcacccatacgcctcccaatactcaagacccgacggccctatcttgctgcaagacttccatctattggagaatatcgctagctttgacagagaaagagtaccagagcgtgtagtccacgccaaaggtggtggttgtagattagaattcgaattgacggattctttgagcgacattacatacgctgctccttaccaagaagtgggttacaaatgccccggccttgtccgtttctccactgttggtggtgaaagtggtaccccggacactgcaagagacccaagaggtgtttctttcaaattctacactaaatggggcaaccacgactgggtctttaataacacacccgtattctttttaagggatgccatcaagttccctgcattcattcacacccaaaagagagacccccaaagtcacctgaatcaattcgaggacactaccctttactgggattatttgactatgaaccccgagtcgattcaccaaatcacatatatgtttggtgagagaggtactcctgcctcttggtctgacatgaacgcttactccggccactcatatatcatggtcaacaaagaggggaaggacacttatgtacagtttcacgtcctgtcggatactggtttcgaaacattgaccggcgacaaagctgctgaactatctggttctcatccagactataaccaggctaagttgttcacacaaatgcaaaacggtgaaaagccaaaattcacctgttatgtacaaacaatgactcctgaacaagccgccaagttcaaatactcggttaatgacttgaccaagatctggccgcacaaagaattcccattgagaaaattcggtaccatcacgttgactactaacgttgacaactactttcaagaaatcgagcaagtagcattcagtccaaccaacacttgtattccaggtattaaaccttccaatgattcagttctacaggctagactcttctcttacccagacacgcaacgacacagactaggggccaactatcagcaactgcctgtcaacagaccaagaaatcttggatgtccatactctaaggaggactcccaatactctgccgaacagtgtccattcaaggctgctaactttcaaagagacggcccaatgagttactataactttggtcctgaaccaaactatctctccagtttaccaaaccaaactctcaaattcaaaaatgaacaaaacgatgaagtgtctgacgaattcaaaggaatagttctagatgaagttaccaaggcttctttgaaaaaacaagaagaagaccaaatcagaaatgaacatatcgtcgatgctaagatcaatcaatattattacgtctacggtatgagtccccttgacttcgaacaacctagagctttatttgaaaaagtttacgaccctgaacaaaagaagctattcgttcataacgttgtttgccatgcttgtaagattaaagatcctaatgtgaaaaagagagttacccaatactttggtttgttgaacgaggacttgggtaaagtcatcgctgagggcctggaggttgaatgggaacctgtcgaccttcaaggttacgccaagacttggagcattgcaagtgcaaactaa

SEQ ID NO: 2 - protein, 560 aa, Saccharomyces sp.
MFGKKEEKPEKVYTLQNGFPYSHHPYASQYSRPDGPILLQDFHLLENIASFDRERVPERVVHAKGGGCRLEFELTDSLSDITYAAPYQEVGYKCPGLVRFSTVGGESGTPDTARDPRGVSFKFYTKWGNHDWVFNNTPVFFLRDAIKFPAFIHTQKRDPQSHLNQFEDTTLYWDYLTMNPESIHQITYMFGERGTPASWSDMNAYSGHSYIMVNKEGKDTYVQFHVLSDTGFETLTGDKAAELSGSHPDYNQAKLFTQMQNGEKPKFTCYVQTMTPEQAAKFKYSVNDLTKIWPHKEFPLRKFGTITLTTNVDNYFQEIEQVAFSPTNTCIPGIKPSNDSVLQARLFSYPDTQRHRLGANYQQLPVNRPRNLGCPYSKEDSQYSAEQCPFKAANFQRDGPMSYYNFGPEPNYLSSLPNQTLKFKNEQNDEVSDEFKGIVLDEVTKASLKKQEEDQIRNEHIVDAKINQYYYVYGMSPLDFEQPRALFEKVYDPEQKKLFVHNVVCHACKIKDPNVKKRVTQYFGLLNEDLGKVIAEGLEVEWEPVDLQGYAKTWSIASAN

SEQ ID NO: 3 - DNA, 1689 bp, Saccharomyces sp.
atgaacgtgttcggtaaaaaagaagaaaagcaagaaaaagtttactctctacaaaacggttttccgtactctcatcacccatacgcttctcaatactcaagaccagacggccctatcttactgcaagacttccatctgctggaaaatatcgcaagtttcgatagagaaagagttccggagcgtgtagtccatgccaaaggtggtggttgtagactggagttcgaactaacagattctttgagtgatattacatacgccgctccataccagaatgtgggttacaaatgtcctggtcttgttcgtttttccaccgttggtggtgaaagtggtacaccagacactgcaagagacccaagaggtgtttcttttaaattctataccgagtgggggaaccatgactgggtcttcaacaatactcccgtcttcttcctcagagacgctattaagtttcccgtatttattcattcgcaaaagagagaccctcagtctcatctgaatcagtttcaggacactaccatatactgggattatctaacattgaatccggaatcaatccatcaaataacttacatgtttggtgatagaggtactcctgcttcgtgggctagtatgaacgcgtactctggtcattccttcatcatggtcaacaaagaaggtaaggacacatatgtgcaattccacgtcttgtcggatactggttttgaaaccttgactggagataaggctgctgaactgtcaggctcccaccctgattataatcaggcaaagctgttcactcaattgcaaaatggcgaaaagccaaaatttaactgttatgtgcaaacaatgacacccgaacaagcaactaagttcaggtattcggtaaatgacctaacgaaaatatggccacacaaggaattccctttgagaaaatttggtaccatcaccctaacggagaatgttgacaattatttccaagaaattgaacaagttgcattcagtccaacgaacacttgtatcccaggtattaagccttctaatgattccgttctacaagccagacttttctcctatccagacactcaacgtcatagattgggagccaactatcaccaattgcctgtcaacagaccaagaaacttgggatgtccatactccaaaggtgattcccaatacactgccgaacagtgtccatttaaagcagtgaacttccaaagggacggcccaatgagttactacaatttcggtcctgagccaaattatatttctagtttaccaaatcaaactctgaaattcaaaaatgaagacaacaacgaagtatctgataagttcaaagggatagttcttgacgaagtaacagaagtttctgtgagaaagcaggaacaagagcaaatcagaaacgagcatattgttgatgccaaaattaatcaatattactacgtttatggtattagtccactagacttcgaacagccaagagctctatatgaaaaggtatacaacgatgaacagaagaaattattcgttcataacgttgtttgccacgcttgtaagatcaaagatcctaaagtcaaaaagagagttacgcaatactttggtttgctaaacgaagatttgggtaaagtcattgcagaaggcttgggagttccttgggaacctgttgaccttgaaggttatgccaagacttggtccattgcaagtgccaattaa

SEQ ID NO: 4 - protein, 562 aa, Saccharomyces sp.
MNVFGKKEEKQEKVYSLQNGFPYSHHPYASQYSRPDGPILLQDFHLLENIASFDRERVPERVVHAKGGGCRLEFELTDSLSDITYAAPYQNVGYKCPGLVRFSTVGGESGTPDTARDPRGVSFKFYTEWGNHDWVFNNTPVFFLRDAIKFPVFIHSQKRDPQSHLNQFQDTTIYWDYLTLNPESIHQITYMFGDRGTPASWASMNAYSGHSFIMVNKEGKDTYVQFHVLSDTGFETLTGDKAAELSGSHPDYNQAKLFTQLQNGEKPKFNCYVQTMTPEQATKFRYSVNDLTKIWPHKEFPLRKFGTITLTENVDNYFQEIEQVAFSPTNTCIPGIKPSNDSVLQARLFSYPDTQRHRLGANYHQLPVNRPRNLGCPYSKGDSQYTAEQCPFKAVNFQRDGPMSYYNFGPEPNYISSLPNQTLKFKNEDNNEVSDKFKGIVLDEVTEVSVRKQEQEQIRNEHIVDAKINQYYYVYGISPLDFEQPRALYEKVYNDEQKKLFVHNVVCHACKIKDPKVKKRVTQYFGLLNEDLGKVIAEGLGVPWEPVDLEGYAKTWSIASAN

SEQ ID NO: 5 - DNA, 40 bp, Artificial
  Primer
gagctcatagcggccatgtttggtaaaaaggaagaaaagc

SEQ ID NO: 6 - DNA, 42 bp, Artificial
  Primer
ggatcctatgcggccgcgtatatatgtaataaataatagtgc

SEQ ID NO: 7 - DNA, 21 bp, Artificial Sequence
  Primer
gtcgacatgaacgtgttcggt

SEQ ID NO: 8 - DNA, 23 bp, Artificial Sequence
  Primer
gcggccgcaatagtgctgcctta